(19) United States
(12) Patent Application Publication
(10) Pub. No.: US 2009/0291112 A1
(43) Pub. Date: Nov. 26, 2009

(54) Allograft osteochondral plug combined with cartilage particle mixture

(76) Inventors: Katherine G. Truncale, Hillsborough, NJ (US); Arthur A. Gertzman, Flemington, NJ (US); Moon Hae Sunwoo, Old Tappan, NJ (US); William W. Tomford, Belmont, MA (US); Judith I. Yannariello-Brown, Somerset, NJ (US); Yen-Chen Huang, East Brunswick, NJ (US)

Correspondence Address: GREENBERG TRAURIG, LLP, 200 PARK AVE., P.O. BOX 677, FLORHAM PARK, NJ, 07932, US

(21) Appl. No.: 12/322,996

(22) Filed: Feb. 9, 2009

Related U.S. Application Data
(63) Continuation-in-part of application No. 10/438,883, filed on May 16, 2003.
Application No. 10/438,883 is a continuation-in-part of application No. 11/151,270, filed on Jun. 14, 2005, now Pat. No. 7,488,348.
Application No. 11/151,270 is a continuation-in-part of application No. 12/179,034, filed on Jul. 24, 2008.
(60) Provisional application No. 61/189,252, filed on Aug. 15, 2008.
(60) Provisional application No. 61/205,433, filed on Jan. 15, 2009.

(51) Int. Cl. A61F 2/28; A61K 38/18; A61K 38/00; A61K 35/32; A61K 35/28; A61K 38/28

(52) U.S. Cl. 424/423; 623/16.11; 514/12; 514/2; 424/93.7; 424/549; 514/3

(57) ABSTRACT

An allograft osteochondral plug is combined with a mixture that includes freeze-milled cartilage particles, and such combination is used to repair defects in articular cartilage. The plug includes an subchondral bone portion and an integral overlying cartilage cap which is treated to remove cellular debris and proteoglycans. At least a portion of the plug has a lateral dimension selected to form an interference fit against a tissue layer exposed as a result of a bore formed in a defect area in articular cartilage of a host. The cartilage particle mixture is placed adjacent at least a portion of the plug for promoting cartilage cell migration into (i.e., from the adjacent host cartilage) and proliferation in the bore, and for enhancing tissue integration between the plug and patient (i.e., host) tissue when the plug is inserted into the bore. Methods for surgical implantation of the plug into a patient are also disclosed.

### RELATED APPLICATIONS

This application is a continuation-in-part of (i) U.S. patent application Ser. No. 10/438,883 filed May 16, 2003, (ii) U.S. patent application Ser. No. 11/151,270 filed Jun. 14, 2005, and (iii) U.S. patent application Ser. No. 12/179,034 filed Jul. 24, 2008, and also claims priority to (iv) U.S. Provisional Patent Application Ser. No. 61/189,252 filed Aug. 15, 2008, and (v) U.S. Provisional Patent Application Ser. No. 61/205,433 filed Jan. 15, 2009. All of the foregoing related applications are incorporated by reference herein in their entirety.

### FIELD OF INVENTION

The present invention is generally directed toward allograft tissue plugs and a cartilage particle mixture for use in repairing defects in articular cartilage. More particularly, the present invention is directed toward allograft osteochondral plugs, each of which includes a subchondral bone base and a cartilage cap, and a cartilage mixture containing freezer-milled (i.e., freeze-milled) cartilage particles. The allograft plug and cartilage particle mixture are used in combination in a bore formed during a surgical procedure to repair an articular cartilage defect.

### BACKGROUND OF THE INVENTION

Chondrogenesis is the process of growth and differentiation of cartilage cells (chondrocytes), leading to the proliferation of such cells and the development of a robust, specialized extracellular matrix surrounding such cells. Cartilage is the specialized matrix of chondrocytes and certain cartilage extracellular matrix components surrounding such chondrocytes. The chondrocytes of damaged cartilage tissue undergo a repair process involving disordered growth that results in tissue with primarily fibrotic morphology, as opposed to the cartilage extracellular matrix resulting from the normal growth and development of chondrocytes and having characteristic proteoglycan and collagen II components.

Articular cartilage injury and degeneration present medical problems to the general population which are constantly addressed by orthopedic surgeons. Thousands of arthroplastic and joint repair procedures are performed every year in the United States, including total hip and total knee arthroplasties and open arthroscopic procedures to repair cartilaginous defects of the knee.

Reference is now made to FIG. 1, which illustrates a knee joint having articular cartilage tissue forming a lining which faces the joint cavity on one side, and is linked to the subchondral bone plate by a narrow layer of calcified cartilage tissue on the other side. Articular cartilage consists primarily of extracellular matrix with a sparse population of chondrocytes distributed throughout the tissue. Articular cartilage is composed of chondrocytes, type II collagen fibril meshwork, proteoglycans, and water. Active chondrocytes are unique in that they have a relatively low turnover rate and are sparsely distributed within the surrounding matrix. The collagen gives the tissue its form and tensile strength and the interaction of proteoglycans with water gives the tissue its stiffness to compression, resilience and durability. The articular cartilage provides a low friction bearing surface over the bony parts of the joint. If the lining becomes worn or damaged, lesions may form in the lining, and as a result, joint movement may become painful or severely restricted. Whereas damaged bone typically can regenerate successfully, articular cartilage regeneration is quite limited because of its limited regenerative and reparative abilities.

Articular cartilage lesions, and other defects, generally do not heal, or heal only partially under certain biological conditions, due to the lack of nerves, blood vessels and a lymphatic system. The limited reparative capabilities of articular cartilage usually results in the generation of repair tissue that lacks the structure and biomechanical properties of normal articular cartilage. Generally, the healing of the lesion results in a fibrocartilaginous repair tissue that lacks the structure and biomedical properties of articular cartilage and degrades over the course of time. Articular cartilage lesions are frequently associated with disability and with symptoms such as joint pain, locking phenomena and reduced or disturbed function. These lesions are difficult to treat because of the distinctive structure and function of articular cartilage. Such lesions are believed to progress to severe forms of osteoarthritis, which is the leading cause of disability and impairment in middle-aged and older individuals, entailing significant economic, social and psychological costs. Each year, osteoarthritis accounts for millions of physician visits and thousands of hospital admissions.

### SUMMARY OF THE INVENTION

A combination for repairing articular cartilage defects includes (i) a mixture including lyophilized, freeze-milled cartilage particles, and (ii) a sterile allograft osteochondral plug. The plug includes a subchondral bone portion and an integral overlying cartilage cap which has been treated to remove cellular debris, chondrocytes and proteoglycans. At least a portion of the plug has a lateral dimension (e.g., diameter) selected to form an interference fit against a tissue layer exposed as a result of a bore formed in a defect area in articular cartilage of a host. The cartilage particle mixture is placed adjacent at least a portion of the plug for promoting cartilage cell migration into and proliferation in the bore and for enhancing tissue integration between the plug and host tissue, when the plug is inserted into the bore. Additives may be included in the cartilage particle mixture in order to increase chondrocyte (and/or other cellular) migration (i.e., from the adjacent host cartilage) and proliferation. Such chondrogenic stimulating additives may include, but are not limited to, growth factors (e.g., FGF-2, FGF-5, FGF-7, FGF-9, FGF-11, FGF-21, TGF-β (including TGF-β1), BMP-2, BMP-4, BMP-7, BMP-14 (GDF-5), PDGF, VEGF, IGF-1), human allogenic or autologous chondrocytes, human allogenic or autologous bone marrow cells, stem cells, demineralized bone matrix, insulin, insulin-like growth factor-1, interleukin-1 receptor antagonist, hepatocyte growth factor, platelet-derived growth factor, Indian hedgehog and parathyroid hormone-related peptide or bioactive glue. Related surgical procedures for repair articular cartilage defect are also disclosed.

### BRIEF DESCRIPTION OF THE DRAWINGS

The patent or application contains at least one drawing executed in color, which includes a color photograph. Copies of this patent or patent application publication with color drawings will be provided by the Office upon request and payment of the necessary fee.

The present invention will be further explained with reference to the attached drawings, wherein like structures are referred to by like numerals throughout the several views. The drawings shown are not necessarily to scale, with emphasis instead generally being placed upon illustrating the principles of the present invention.

FIG. 1 is an anatomical illustration of a knee joint having an articular cartilage surface in which a lesion has formed;

FIG. 2 is a top perspective view of an allograft osteochondral plug produced in accordance with an embodiment of the present invention and positioned in an articular cartilage defect site;

FIG. 3 is a top perspective view of the osteochondral plug of FIG. 2;

FIG. 4 is a top perspective view of an allograft osteochondral plug produced in accordance with another embodiment of the present invention and positioned in an articular cartilage defect site;

FIG. 5 is a top perspective view of the osteochondral plug of FIG. 4;

FIG. 6 is a top perspective view of an allograft osteochondral plug produced in accordance with another embodiment of the present invention and positioned in an articular cartilage defect site;

FIG. 7 is a top perspective view of the osteochondral plug of FIG. 6;

FIG. 8 is a schematic illustration showing a cross-sectional view of a blind bore formed in an articular cartilage defect site in connection with the implantation of the osteochondral plug of FIG. 6;

FIG. 9 is a top perspective view of an osteochondral plug produced in accordance with another embodiment of the present invention;

FIG. 10 is a top perspective view of an allograft osteochondral plug produced in accordance with another embodiment of the present invention and positioned in an articular cartilage defect site;

FIG. 11 is a top perspective view of the allograft osteochondral plug of FIG. 10;

FIG. 12 is a top perspective view of an allograft osteochondral plug produced in accordance with another embodiment of the present invention;

FIG. 13 is a top perspective view of an allograft osteochondral plug produced in accordance with another embodiment of the present invention;

FIG. 14 is a depiction of nanograms of endogenous TGF-β 1 per gram of cartilage particles isolated from said cartilage particles of several subjects through guanidine HCl extraction and subsequent dialysis;

FIG. 15 is a comparison of relative amounts (nanograms) of endogenous TGF-β1 per gram of cartilage particles isolated from minced and freeze-milled cartilage through guanidine HCl extraction and subsequent dialysis;

FIG. 16 is a depiction of picograms of endogenous FGF-2 per gram of cartilage particles isolated from said cartilage particles of several subjects through guanidine HCl extraction and subsequent dialysis;

FIG. 17 is a depiction of nanograms of endogenous BMP-2 per gram of cartilage particles isolated from said cartilage particles of several subjects through guanidine HCl extraction and subsequent dialysis;

FIG. 18 is a depiction of nanograms of endogenous BMP-14 (GDF-5) per gram of cartilage particles isolated from said cartilage particles of several subjects through guanidine HCl extraction and subsequent dialysis;

FIG. 19 is a depiction of nanograms of endogenous IFG-1 per gram of cartilage particles isolated from said cartilage particles of several subjects through guanidine HCl extraction and subsequent dialysis;

FIG. 20 is a view of newly synthesized articular cartilage from in vivo experimentation, demonstrating infiltration of lacunae by chondrocytes; and

FIGS. 21A and 21B are views of collagen immunohistochemistry staining for collagen II, a marker of articular cartilage, showing that both cartilage particles and newly-synthesized extracellular matrix stain positive for collagen II.

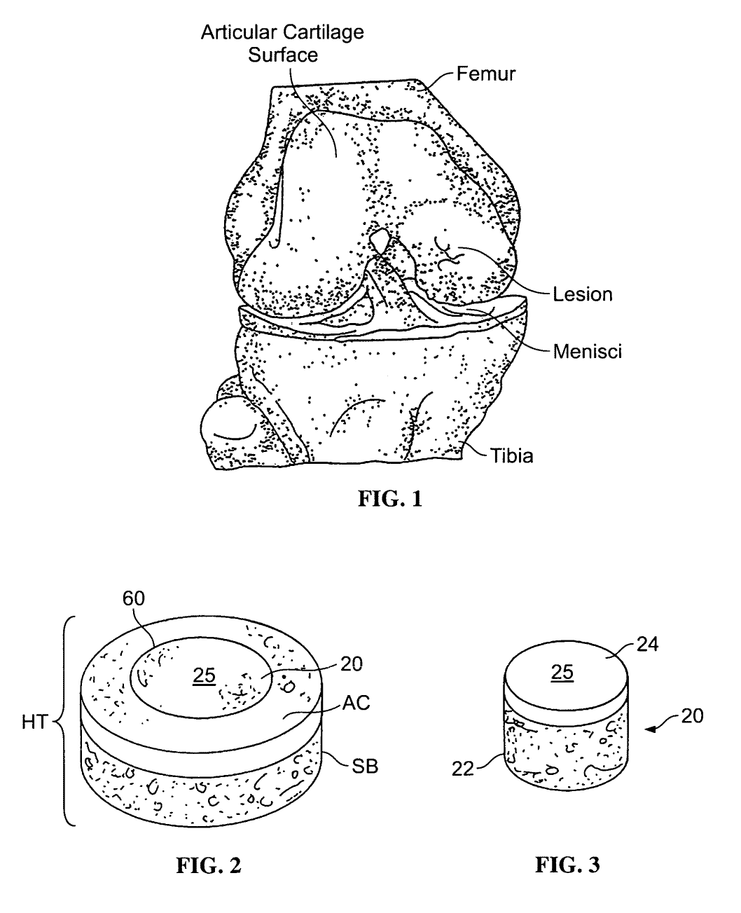

While the above-identified drawings set forth presently disclosed embodiments of the present invention, other embodiments are also contemplated, as noted in the detailed description. This disclosure presents illustrative embodiments by way of representation and not limitation. Numerous other modifications and embodiments can be devised by those skilled in the art and all such modifications and embodiments fall within the scope and spirit of the principles of the presently disclosed invention.

### DETAILED DESCRIPTION OF THE INVENTION

While detailed embodiments of the present invention are disclosed herein, it is to be understood that the disclosed embodiments are merely illustrative of the invention that may be embodied in various forms. In addition, each of the examples given in connection with the various embodiments of the invention are intended to be illustrative, and not restrictive. Further, the figures are not necessarily to scale, some features may be exaggerated to show details of particular components. In addition, any measurements, specifications and the like shown in the figures are intended to be illustrative, and not restrictive. Therefore, specific structural and functional details disclosed herein are not to be interpreted as limiting, but merely as a representative basis for teaching one skilled in the art to variously employ the present invention.

### Osteochondral Plugs

One embodiment of an allograft osteochondral plug 20 is illustrated in FIGS. 2 and 3. The plug 20 is recovered from a donor and is then treated to remove cellular material, chondrocytes, pluripotent mesenchymal cells and proteoglycans according to the following procedure. The plug 20 (alone or with other allograft osteochondral plugs) is soaked in a solution or variety of solutions, such as hyaluronidase (type IV-S, 3 mg/mL), trypsin (0.25% in monodibasic buffer 3 ml) and a chloroform/methanol solution, to remove the cellular debris and the proteoglycans. The plug 20 is then placed in a test tube for 18 hours at 37° C. with sonication. Alternatively, the plug 20 may be treated without sonication. Soaking times may vary with the concentration of the solutions used, and can be as little as two (2) hours. It is believed that removal of the cellular debris and the proteoglycans provides signaling to stimulate the surrounding chondrocytes to proliferate and form new proteoglycans and other factors to produce new cartilage matrix. The above treatment method has been previously used on human tissue and is set forth in the Journal of Rheumatology, 12:4, (1985) by Gust Verbruggen et al., titled “Repair Function in Organ Cultured Human Cartilage Replacement of Enzymatically Removed Proteoglycans During Longterm Organ Culture”, which is incorporated by reference herein. Other treatment methods yielding similar results are known to persons skilled in this particular art.

Next, the plug 20 is subjected to an antibiotic soak and milled to a desired configuration (e.g., to have an interference fit with the blind bore to be cut in the patient's tissue), such as that illustrated in FIG. 3. After repeated washes with sterile DI water, the hydrated plug 20 is frozen at −20° C. to −100° C., preferably at −70° C., and lyophilized (i.e., freeze-dried) to reduce the water content within a range of about 0.1% to about 8.0%. The lyophilized plug 20 is then secured in an appropriate moisture barrier package for long-term storage, whereby the lyophilized plug 20 may be stored at room temperature for up to five years. Examples of moisture barrier packaging that may be used include a flexible foil laminate pouch and a high moisture barrier thermoformed tray heat sealed to foil lidstock. The pouch may be made of materials that can be laminated with foil (e.g., PET, PE, LDPE, LLDPE, HDPE, Nylon), while the tray may be made of a laminate material (e.g., PETG/PCTFE laminate, PVC/PCTFE laminate, PETG/COC laminate, PVC/COC laminate, COC/PCTFE laminate). To preserve the sterility of the lyophilized plug 20, packaging with sterile barrier properties is used. The package may consist of more than one layer to facilitate the transfer of the plug 20 into the sterile field of an operating room or other sterile environments.

Alternatively, the plug 20 may be frozen at −20° C. to −100° C., preferably at −70° C., (i.e., without lyophilization), whereby the frozen plug 20 may be stored at the aforementioned temperature(s) for up to five years. The frozen plug 20 may be stored in a multiple-layered moisture barrier package to maintain sterility, as discussed above.

Plugs 20 that have been processed and stored as described above are produced in various standard sizes (i.e., diameters and heights). Prior to a surgical articular cartilage repair procedure, a surgeon may pre-order a set of plugs 20 for use in connection with the surgery. During surgery, a surgeon selects one of the plugs 20 having a diameter that matches the diameter of a cylindrical hole (i.e., a blind bore) 60 that has been cut in the lesion area of the host tissue HT (i.e., the subchondral bone SB and the overlying articular cartilage AC) of a patient, and inserts the plug 20 into the bore 60 (see FIG. 2). The plug 20 that is selected by the surgeon will have a diameter sized to facilitate an interference fit between the plug 20 and a sidewall of the bore 60. The plug 20 has a cylindrical subchondral bone portion 22 and an overlying, integral, disc-shaped cartilage cap 24 having a diameter that is substantially equal to the diameter of the bone portion 22 (see FIG. 3).

In one embodiment, the plug 20 has a height that is substantially equal to the depth of the bore 60, wherein the plug 20 is supported by a bottom surface of the bore 60. This type of load-bearing support protects the plug 20 from damage caused by micromotion at the interface of the bore 60 and the plug 20, which may produce undesired fibrous tissue interfaces and subchondral cysts.

In an alternative embodiment, the height of the plug 20 may be less than the bore depth. In this embodiment, the plug 20 is supported by the sidewall of the bore 60 due to the aforementioned interference fit within the bore 60. This type of load-bearing support also protects the plug 20 from the aforementioned damage caused by micromotion at the interface of the bore 60 and the plug 20.

In another embodiment, if the depth of the bore 60 is less than the height of the plug 20, the surgeon may match the height of the plug 20 to the bore depth by removing tissue from the bottom of the plug 20. More particularly, the surgeon may cut away a bottom portion of the subchondral bone portion 22 to decrease the height of the plug 20 and thereby match the plug 20 to the depth of the bore 60.

The height of the plug 20 may determine its placement in the bore 60, and, hence, the position of an upper surface 25 of the cartilage cap 24 in relation to the surface of the adjacent host cartilage AC. The plug 20 may be placed in the bore 60 so that the upper surface 25 is substantially flush with the surface of the adjacent host cartilage AC of the host tissue HT in the articulating joint, thereby forming a smooth, continuous load-bearing surface (see FIG. 2). Alternatively, the plug 20 may be placed so that the upper surface 25 is slightly higher than the surface of the adjacent host cartilage AC, so as to be proud in relation thereto. The plug 20 may also be placed so that the upper surface 25 is slightly lower than the surface of the adjacent host cartilage AC, thereby providing a space, or pocket, for tissue growth within such space. A cartilage particle mixture may also be placed in the space. The cartilage particle mixture promotes chondrocyte (and/or other cellular) migration into (i.e., from the adjacent host cartilage AC) and proliferation in the bore 60, and enhances tissue integration between the cartilage cap 24 and the adjacent host articular cartilage AC. The cartilage particle mixture, which may contain freezer-milled (i.e., freeze-milled) cartilage particles, may be in the form of a paste or gel, is described in greater detail below.

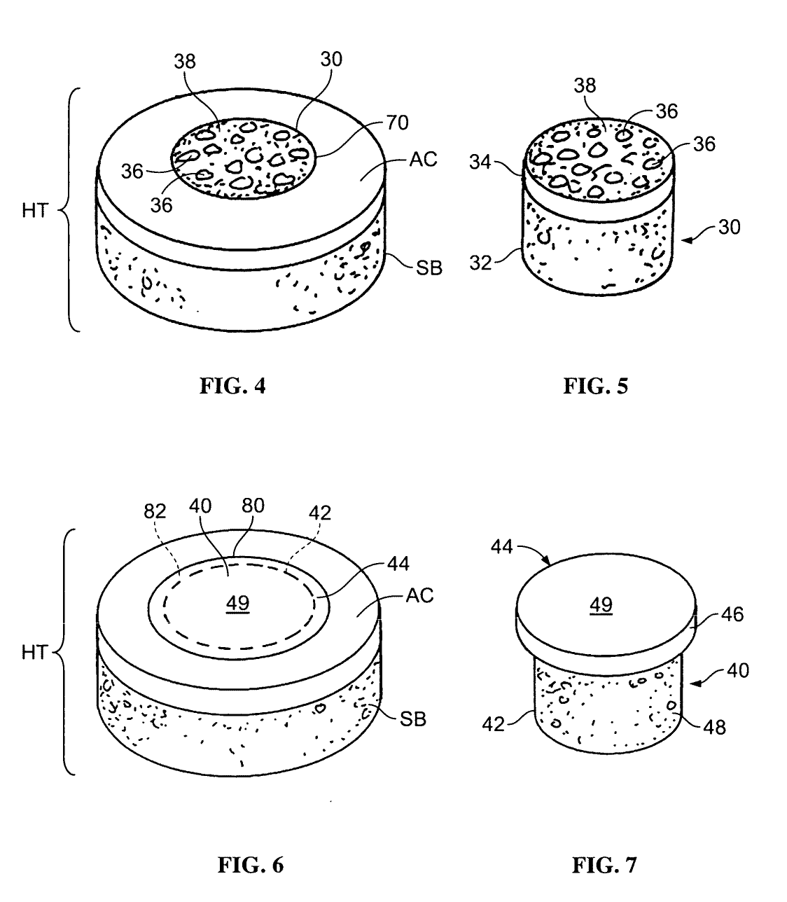

Another embodiment of an allograft osteochondral plug 30 is illustrated in FIGS. 4 and 5. The plug 30 may be treated according to the treatment described above in connection with the plug 20 (i.e., decellularizing and cleaning the plug). The plug 30 may then be lyophilized or frozen, and packaged for long-term storage, as described above in connection with the plug 20. The modifications in the size and surgical insertion of the plug 20 described above may be made in connection with the plug 30 as well.

Plugs 30 that have been processed and stored as described above are produced in various standard sizes (i.e., diameters and heights). Prior to a surgical articular cartilage repair procedure, a surgeon may pre-order a set of plugs 30 for use in connection with the surgery. During surgery, a surgeon selects one of the plugs 30 having a diameter that matches the diameter of a cylindrical hole (i.e., a blind bore) 70 that has been cut in the lesion area of the host tissue HT (i.e., the subchondral bone SB and the overlying articular cartilage AC) of a patient (see FIG. 4). The plug 30 that is selected by the surgeon will have a diameter sized to facilitate an interference fit between the plug 30 and a sidewall of the bore 70. The plug 30 has a cylindrical subchondral bone portion 32 and an overlying, integral, disc-shaped cartilage cap 34 having a diameter that is substantially equal to the diameter of the bone portion 32 (see FIG. 5). Unless indicated otherwise below, the bone portion 32 and the cartilage cap 34 are constructed in the same general manner as the bone portion 22 and the cartilage cap 24, respectively, of the plug 20 illustrated in FIGS. 2 and 3.

Still referring to FIGS. 4 and 5, a plurality of throughgoing bores 36 is drilled through the cartilage cap 34 and the bone portion 32. The throughgoing bores 36 facilitate the migration, or upward passage, of bone marrow derived from stems cells from the bleeding bone of the patient. The throughgoing bores 36 also facilitate the migration, or upward passage, of a cartilage particle mixture which may be placed in the bore 70 prior to the insertion of the plug 30. More particularly, when the surgeon places the plug 30 into the bore 70, it compresses the cartilage particle mixture therein and causes it to move from a bottom wall of the bore 70 (i.e., adjacent the bone portion 32) upwards, through the throughgoing bores 36, and adjacent the cartilage cap 34 and an adjacent upper portion of the bore. The cartilage particle mixture promotes chondrocyte (and/or other cellular) migration into (i.e., from the adjacent host cartilage AC) and proliferation in the bore 70, and enhances tissue integration between the cartilage cap 34 and the adjacent host articular cartilage AC. The cartilage particle mixture is described in greater detail below.

The height of the plug 30 may determine its placement in the bore 70, and, hence, the position of an upper surface 38 of the cartilage cap 34 in relation to the surface of the adjacent cartilage AC. The plug 30 may be placed in the bore 70 so that the upper surface 38 is substantially flush with the surface of the adjacent host cartilage AC (see FIG. 4). Alternatively, the plug 30 may be placed so that the upper surface 38 is slightly higher than the surface of the adjacent host cartilage AC so as to be proud in relation thereto. The plug 30 may also be placed so that the upper surface 38 is slightly lower than the surface of the adjacent host cartilage AC, thereby providing space, or a pocket, for tissue growth within such space. The cartilage particle mixture described herein may also be placed in the space. The cartilage particle mixture promotes chondrocyte (and/or other cellular) migration into and proliferation in the bore 70, and enhances tissue integration between the cartilage cap 34 and the adjacent host articular cartilage AC.

Another embodiment of an allograft osteochondral plug 40 is illustrated in FIGS. 6 and 7. The plug 40 may be treated according to the treatment described above in connection with the plug 20 (i.e., decellularizing and cleaning the plug). The plug 40 may then be lyophilized or frozen, and packaged for long-term storage, as described above in connection with the plug 20. The modifications in the size and surgical insertion of the plug 20 described above may be made in connection with the plug 40 as well.

Plugs 40 that have been processed and stored as described above are produced in various standard sizes (i.e., diameters and heights). The plug 40 has a cylindrical subchondral bone portion 42 and an overlying, integral disc-shaped cartilage cap 44. The cartilage cap 44 has a larger diameter than that of the bone portion 42. More particularly, a peripheral rim 46 of the cap 44 extends past a cylindrical sidewall 48 of the bone portion 42, thereby imparting a mushroom shape to the plug 40 (see FIG. 7).

Prior to a surgical articular cartilage repair procedure, a surgeon may pre-order a set of plugs 40 for use in connection with the surgery. During surgery, a surgeon selects one of the plugs 40 with a subchondral bone portion 42 having a diameter that matches the diameter of a cylindrical hole (i.e., a blind bore) 80 that has been cut in the lesion area of the host tissue HT (i.e., the subchondral bone SB and the overlying articular cartilage AC) of a patient (see FIG. 6). The subchondral bone portion 42 of the plug 40 that is selected by the surgeon will have a diameter sized to facilitate an interference fit between the subchondral bone portion 42 and a sidewall of the bore 80, as further described below.

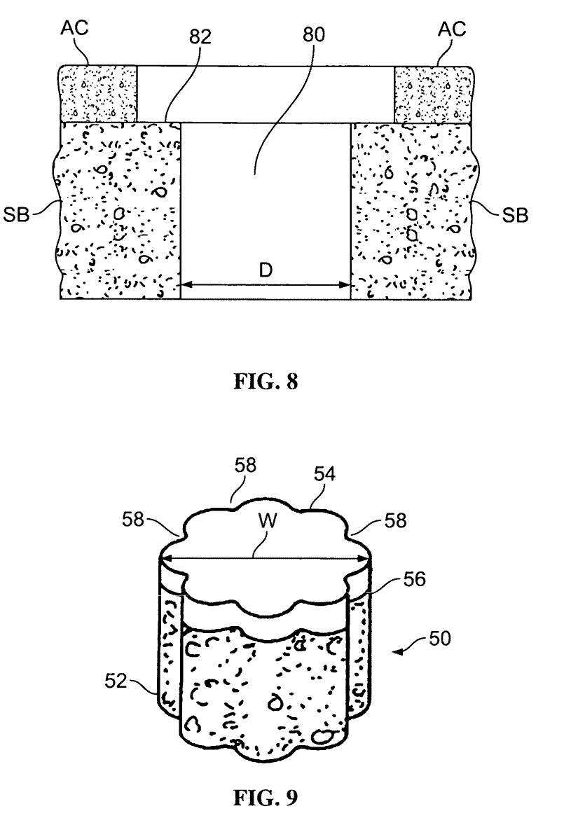

Prior to placing the plug 40 into the bore 80, the bore 80 is countersunk to the depth of the host cartilage AC, as shown in FIG. 8, thereby forming an annular ledge 82 that supports a lower surface (not visible) of the rim 46 such that an upper surface 49 of the cartilage cap 44 is flush with the surface of the adjacent host cartilage AC (see FIG. 6). After forming the annular ledge 82, the plug 40 is placed in the bore 80. In this embodiment, the diameter of the subchondral bone portion 42 matches the diameter D of the bore 80, facilitating an interference fit between the subchondral bone portion 42 and a sidewall of the bore 80.

The height of the plug 40 may determine its placement in the bore 80, and, hence, the position of an upper surface 49 of the cartilage cap 44 in relation to the surface of the adjacent host cartilage AC. The subchondral bone portion 42 has a height which engages the floor of the bore 80 so that the upper surface 49 is flush with the surface of the adjacent host cartilage AC of the articulating joint to form a smooth, continuous load-bearing surface. Alternatively, the plug 40 may be placed so that the upper surface 49 is slightly higher than the surface of the adjacent host cartilage AC, so as to be proud in relation thereto. The plug 40 may also be placed so that the upper surface 49 is slightly lower than the surface of the adjacent host cartilage AC, which provides a space, or pocket, for tissue growth within such space. The cartilage particle mixture described herein may also be placed in the space. The cartilage particle mixture promotes chondrocyte (and/or other cellular) migration into (i.e., from the adjacent host cartilage AC) and proliferation in the bore 80, and enhances tissue integration between the cartilage cap 44 and the adjacent host articular cartilage AC.

In another embodiment (not illustrated), no annular ledge 82 is formed, the diameter of the cartilage cap 44 matches the diameter D of the bore 80, and the diameter of the subchondral bone portion 42 is smaller than the diameter of the cartilage cap 44. Because the diameter of the subchondral bone portion 42 is also smaller than the diameter D of the bore 80, there is no interference fit between the subchondral bone portion 42 and the bore 80. Consequently, the subchondral bone portion 42 is spaced from the host subchondral bone SB adjacent the bore 80, thereby forming a ring-shaped gap or trough between the subchondral bone portion 42 and an adjacent portion of the sidewall of the bore 80. The dimensions of the channel may be within a range that is dependent upon such factors as the dimensions of the subchondral bone portion 42 and the dimensions of the bore 80, as well as other factors. A demineralized bone matrix mixture, bone chips, and/or other osteogenic additives known in the art may be placed in the trough to promote bone growth in the trough and to enhance tissue integration between the subchondral bone portion 42 and the surrounding host subchondral bone SB.

Another embodiment of an allograft osteochondral plug 50 is illustrated in FIG. 9. The plug 50 may be treated according to the treatment described above in connection with the plug 20 (i.e., decellularizing and cleaning the plug). The plug 50 may then be lyophilized or frozen, and packaged for long-term storage, as described above in connection with the plug 20. The modifications in the size and surgical insertion of the plug 20 described above may be made in connection with the plug 50 as well.

Plugs 50 that have been processed and stored as described above are produced in various standard sizes (i.e., widths, lateral dimensions and heights). Prior to a surgical articular cartilage repair procedure, a surgeon may pre-order a set of plugs 50 for use in connection with the surgery. During surgery, a surgeon selects one of the plugs 50 having a width W, or maximum lateral dimension, that matches the diameter of a cylindrical hole (i.e., a blind bore) that has been cut in the lesion area of the host tissue (i.e., the subchondral bone and the overlying articular cartilage) of a patient. The plug 50 that is selected by the surgeon will have a width W, or maximum lateral dimension, sized to facilitate an interference fit between the plug 50 and a sidewall of the bore. The plug 50 has a subchondral bone portion 52 and an overlying, integral cartilage cap 54. An exterior sidewall 56 of the plug 50 is provided with a plurality of longitudinal grooves (i.e., channels) 58, each of which extends continuously along the contiguous lengths of the cartilage cap 54 and the subchondral bone portion 52. Alternatively, the channels 58 may only extend along the surface of the subchondral bone portion 52 only ending at a bottom surface of the cartilage cap 54, so as to provide the cartilage cap 54 with a shape similar to that shown in FIG. 7.

The channels 58 facilitate the migration, or upward passage, of the cartilage particle mixture which may be placed in the bore prior to the insertion of the plug 50. More particularly, when the surgeon places the plug 50 into the bore, it compresses the cartilage particle mixture therein and causes it to move from a bottom wall of the bore (i.e., adjacent the subchondral bone portion 52) upwards, through the channels 58, and adjacent the cartilage cap 54 and an adjacent upper portion of the bore. The cartilage particle mixture promotes chondrocyte (and/or other cellular) migration into (i.e., from the adjacent host cartilage) and proliferation in the bore, and enhances tissue integration between the cartilage cap 54 and the adjacent host articular cartilage AC. The cartilage particle mixture is described in greater detail below.

In one surgical procedure to implant an osteochondral plug according to an embodiment of the present invention, such as one of the plugs 30 and 50 disclosed above, an articular cartilage lesion or defect is removed by cutting a bore in the defect area. The bore is filled with a desired amount of the cartilage particle mixture, such as described below. An osteochondral plug according to an embodiment of the present invention, such as one of the plugs 30 or 50 disclosed above, is then placed in the bore (i.e., on top of the cartilage particle mixture) in an interference fit with the sidewall of the bore. This arrangement retains the cartilage particle mixture in the bore for a predetermined period of time, to promote chondrocyte (and/or other cellular) migration into and proliferation in the defect site and enhance tissue integration between the plug and the host surrounding articular cartilage. As explained above, when the cartilage particle mixture is compressed by the plug 30, the mixture moves upwards, through the throughgoing bores 36, and adjacent the cartilage cap 34 and an adjacent upper portion of the bore. Similarly, when the cartilage particle mixture is compressed by the plug 50, the mixture moves upwards, through the channels 58, and adjacent the cartilage cap 54 and an adjacent upper portion of the bore. The cartilage particle mixture may also or alternatively be placed directly on one or more surfaces of the plug during the surgical procedure.

In another surgical procedure to implant an osteochondral plug according to another embodiment of the present invention, such as one of the plugs 20 and 40 disclosed above, an articular cartilage lesion or defect is removed by cutting a bore in the defect area. An osteochondral plug according to an embodiment of the present invention, such as one of the plugs 20 or 40 disclosed above, is then placed in the bore in an interference fit with the sidewall of the bore. The cartilage particle mixture may then be spread on the top surface of the implanted plug. This arrangement also retains the cartilage particle mixture in the bore for a predetermined period of time, to promote chondrocyte (and/or other cellular) migration into and proliferation in the defect site and enhance tissue integration between the plug and the host surrounding articular cartilage. The cartilage particle mixture may also or alternatively be placed on one or more other surfaces of the plug during the surgical procedure.

Suitable organic glue material can be used to keep the plug fixed in place in the bore. Suitable organic glue material can be found commercially, such as for example; TISSEEL® or TISSUCOL®) (fibrin based adhesive; Immuno AG, Austria), Adhesive Protein (Sigma Chemical, USA), Dow Corning Medical Adhesive B (Dow Corning, USA), fibrinogen thrombin, elastin, collagen, casein, albumin, keratin and the like.

The diameter, or lateral dimension, of the plugs according to embodiments illustrated in FIGS. 2-9 may be in a range from 1 mm to 30 mm, but is preferably 4 mm to 10 mm, which is small enough to fit through an endoscopic cannula, but large enough to minimize the number of plugs needed to fill large articular cartilage defects. This size provides good results at the defect area site and provides a more confluent articular cartilage surface. A surgeon may reduce the height of the plug by cutting and removing a bottom portion of the subchondral bone portion to match the depth of the bore, so that the cartilage cap of the plug will be even with the surface of the host articular cartilage.

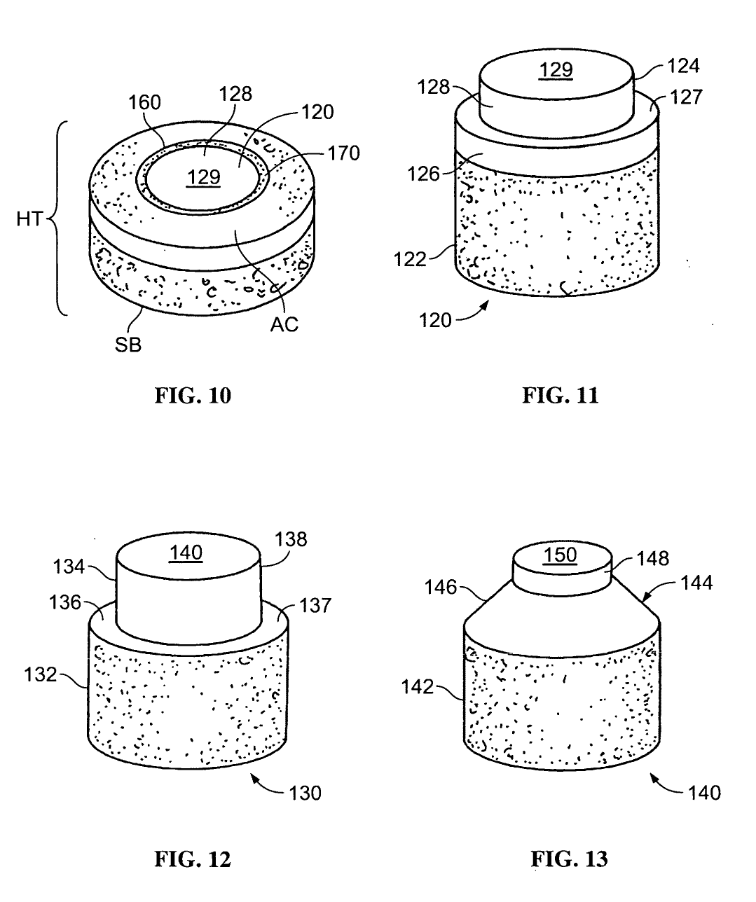

Reference is now made to FIGS. 10 and 11, which illustrate another embodiment of an osteochondral plug 120. Like the other osteochondral plus described above, the plug 120 may be treated according to the treatment described above in connection with the plug 20 (i.e., decellularizing and cleaning the plug). The plug 120 may then be lyophilized or frozen, and packaged for long-term storage, as described above in connection with the plug 20.

Plugs 120 that have been processed and stored as described above are produced in various standard sizes (i.e., diameters and heights). The plug 120 has a subchondral bone portion 122 and an overlying integral cartilage cap 124 (see FIG. 11). An annular upper portion of the cartilage cap 124 is cut away to form a stepped configuration. More particularly, the cartilage cap 124 includes: (i) a lower cap portion 126 which is proximal to the subchondral bone portion 122 and which is provided with an annular top surface (i.e., a step) 127; and (ii) and an upper cap portion 128 which is distal to the subchondral bone portion 122. The lower cap portion 126 has a diameter that is substantially the same as that of the subchondral bone portion 122, while the upper cap portion 128 has a diameter that is smaller than that of the lower cap portion 126, thereby forming the annular ledge 127. In one embodiment, the upper cap portion 128 has a diameter that is in a range from about 200 microns to about 1,000 microns less than the diameter of the subchondral bone portion 122.

Prior to a surgical articular cartilage repair procedure, a surgeon may pre-order a set of plugs 120 for use in connection with the surgery. During surgery, a surgeon selects one of the plugs 120 with the subchondral bone portion 122 having a diameter that matches the diameter of a cylindrical hole (i.e., a blind bore) 160 that has been cut in the lesion area of the host tissue HT (i.e., the subchondral bone SB and the overlying articular cartilage AC) of a patient (see FIG. 10). The subchondral bone portion 122 of the plug 120 that is selected by the surgeon will have a diameter sized to facilitate an interference fit between the subchondral bone portion 122 and a sidewall of the bore 160, as further described below. The plug 120 may be placed in the bore 160 so that a top surface 129 of the upper cap portion 128 is level with (i.e., substantially coplanar in relation to) the surface of the adjacent host cartilage AC of the articulating joint (see FIG. 10).

In one embodiment, the plug 120 has a height that is substantially equal to the depth of the bore 160, wherein the plug 120 is supported by a bottom surface of the bore 160. This type of load-bearing support protects the plug 120 from damage caused by micromotion at the interface of the bore 160 and the subchondral bone portion 122 of the plug 120, which may produce undesired fibrous tissue interfaces and subchondral cysts.

In an alternative embodiment, the height of the plug 120 may be less than the bore depth, wherein the plug 120 is supported by a sidewall of the bore 160 due to the aforementioned interference fit between the sidewall of the bore 160 and the subchondral bone portion 122. This type of load-bearing support also protects the plug 120 from the aforementioned damage caused by micromotion at the interface of the bore 160 and the plug 120.

In another embodiment, if the depth of the bore 160 is less than the height of the plug 120, the surgeon may match the height of the plug 120 to the bore depth by removing tissue from the bottom of the plug 120. More particularly, the surgeon may cut away a bottom portion of the subchondral bone portion 122 to decrease the height of the plug 120 and thereby match the plug 120 to the depth of the bore 160.

The height of the plug 120 may determine its placement in the bore 160, and, hence, the position of an upper surface 129 of the cartilage cap 124 in relation to the surface of the adjacent cartilage AC. The plug 120 may be placed in the bore 160 so that the upper surface 129 is substantially flush with the surface of the adjacent host cartilage AC (see FIG. 10). Alternatively, the plug 120 may be placed so that the upper surface 129 is slightly higher than the surface of the adjacent host cartilage AC, so as to be proud in relation thereto. The plug 120 may also be placed so that the upper surface 129 is slightly lower than the surface of the adjacent host cartilage AC.

Because the upper cap portion 128 of the cartilage cap 124 has a smaller diameter than that of the bore 160, there is no interference fit between the upper cap portion 128 of the cartilage cap 124 and the bore 160. Consequently, the upper cap portion 128 is spaced from the host cartilage AC adjacent the bore 160, thereby forming a ring-shaped gap or trough 170 between the upper cap portion 128 and an adjacent portion of the sidewall of the bore 160 (see FIG. 10). The width of the trough 170 may be in a range between 10 microns and 1,000 microns, and more preferably, between 100 microns and 500 microns. The depth of the channel 170 may be within a range that is dependent upon such factors as the dimensions of the cartilage cap 124 and the dimensions of the bore 160, as well as other factors. The aforementioned cartilage particle mixture (as described below) may be placed in the trough 170 to promote chondrocyte (and/or other cellular) migration into (i.e., from the adjacent host cartilage AC) and proliferation in the trough 170 and to enhance tissue integration between the cartilage cap 124 and the surrounding host articular cartilage AC.

Another embodiment of an osteochondral plug 130 is illustrated in FIG. 12. The plug 130 may be treated according to the treatment described above in connection with the plug 20 (i.e., decellularizing and cleaning the plug). The plug 130 may then be lyophilized or frozen, and packaged for long-term storage, as described above in connection with the plug 20. The modifications in the size and surgical insertion of the plug 120 described above may be made in connection with the plug 130 as well.

Plugs 130 that have been processed and stored as described above are produced in various standard sizes (i.e., diameters and heights). The plug 130 has a subchondral bone portion 132 and an overlying integral cartilage cap 134 (see FIG. 12). An annular upper portion of the cartilage cap 134 is cut away to form a stepped configuration. More particularly, the cartilage cap 134 includes: (i) a lower cap portion 136 which is proximal to the subchondral bone portion 132 and which is provided with an annular top surface (i.e., a step 137); and (ii) and an upper cap portion 138 which is distal to the subchondral bone portion 132. The lower cap portion 136 has a diameter that is substantially the same as that of the subchondral bone portion 132, while the upper cap portion 138 has a diameter that is smaller than that of the lower cap portion 136, thereby forming the annular ledge 137. In one embodiment, the upper cap portion 138 has a diameter that is in a range from about 10 microns to about 1,000 microns less than the diameter of the subchondral bone portion 132. In another embodiment, the upper cap portion 138 has a diameter that is in a range from about 100 microns to about 500 microns less than the diameter of the subchondral bone portion 132.

Prior to a surgical articular cartilage repair procedure, a surgeon may pre-order a set of plugs 130 for use in connection with the surgery. During surgery, a surgeon selects one of the plugs 130 with the subchondral bone portion 132 having a diameter that matches the diameter of a cylindrical hole (i.e., a blind bore) that has been cut in the lesion area of the host tissue (i.e., the subchondral bone and the overlying articular cartilage) of a patient. The subchondral bone portion 132 of the plug 130 that is selected by the surgeon will have a diameter sized to facilitate an interference fit between the subchondral bone portion 132 and a sidewall of the bore, as further described below. The plug 130 may be placed in the bore so that a top surface 140 of the upper cap portion 138 is level with (i.e., substantially coplanar in relation to) the surface of the adjacent host cartilage AC of the articulating joint.

Because the upper cap portion 138 has a smaller diameter than that of the bore, there is no interference fit between the upper cap portion 138 of the cartilage cap 134 and the bore. Consequently, the upper cap portion 138 is spaced from the host cartilage adjacent the bore, thereby forming a ring-shaped gap or trough between the upper cap portion 138 and an adjacent portion of the sidewall of the bore. The width of the trough may be in a range of between 10 microns and 1,000 microns, and more preferably, between 100 microns and 500 microns. The depth of the trough may be within a range that is dependent on such factors as the dimensions of the cartilage cap 134 and the dimensions of the bore, as well as other factors. The aforementioned cartilage particle mixture (as described below) may be placed in the trough to promote chondrocyte (and/or other cellular) migration into (i.e., from the adjacent host cartilage) and proliferation in the trough and to enhance tissue integration between the cartilage cap 134 and the surrounding host articular cartilage.

Another embodiment of an osteochondral plug 140 is illustrated in FIG. 13. The plug 140 may be treated according to the treatment described above in connection with the plug 20 (i.e., decellularizing and cleaning the plug). The plug 140 may then be lyophilized or frozen, and packaged for long-term storage, as described above in connection with the plug 20. The modifications in the size and surgical insertion of the plug 120 described above may be made in connection with the plug 140 as well.

Plugs 140 that have been processed and stored as described above are produced in various standard sizes (i.e., diameters and heights). The plug 140 has a subchondral bone portion 142 and an overlying integral cartilage cap 144 (see FIG. 13). An annular upper portion of the cartilage cap 144 is cut away to form a stepped configuration. More particularly, the cartilage cap 144 includes: (i) a frustum conical lower cap portion 146 which is proximal to the subchondral bone portion 142; and (ii) a cylindrical upper cap portion 148 which is distal to the subchondral bone portion 142. The lower cap portion 146 tapers such that (i) an upper end thereof has a diameter that is slightly greater than the diameter of the upper cap portion 148, and (ii) a lower end thereof has a diameter that is slightly smaller than the diameter of the bore. The lower end diameter may alternatively match the diameter of the bore. The upper cap portion 148 has a smaller diameter than that of the bore. In one embodiment, the upper cap portion 148 has a diameter that is in a range from about 10 microns to about 1,000 microns less than the diameter of the subchondral bone portion 142. In another embodiment, the upper cap portion 148 has a diameter that is in a range from about 100 microns to about 500 microns less than the diameter of the subchondral bone portion 142.

Prior to a surgical articular cartilage repair procedure, a surgeon may pre-order a set of plugs 140 for use in connection with the surgery. During surgery, a surgeon selects one of the plugs 140 with the subchondral bone portion 142 having a diameter that matches the diameter of a cylindrical hole (i.e., a blind bore) that has been cut in the lesion area of the host tissue (i.e., the subchondral bone and the overlying articular cartilage) of a patient. The subchondral bone portion 142 of the plug 140 that is selected by the surgeon will have a diameter sized to facilitate an interference fit between the subchondral bone portion 142 and a sidewall of the bore, as further described below. The plug 140 may be placed in the bore so that a top surface 150 of the upper cap portion 148 is level with (i.e., substantially coplanar in relation to) the surface of the adjacent host cartilage AC of the articulating joint.

Because the upper cap portion 148 has a smaller diameter than that of the bore, there is no interference fit between the upper cap portion 148 of the cartilage cap 144 and the bore. There is also no interference fit between at least a part of the lower cap portion 146 (e.g., the entire lower cap portion 146 except for its lower end) and the bore. Consequently, at least a large part of the cartilage cap 144 (i.e., the upper cap portion 148 and the entire lower cap portion 146 except for its lower end) is spaced from the host cartilage adjacent the bore, thereby forming a ring-shaped gap or trough between the upper cap portion 148 and an adjacent portion of the sidewall of the bore. The width of the trough may be in a range of between 10 microns and 1,000 microns, and more preferably, between 100 microns and 500 microns. The depth of the trough may be within a range that is dependent on such factors as the dimensions of the cartilage cap 144 and the dimensions of the bore, as well as other factors. The aforementioned cartilage particle mixture (as described below) may be placed in the trough to promote chondrocyte (and/or other cellular) migration into (i.e., from the adjacent host cartilage) and proliferation in the trough and to enhance tissue integration between the cartilage cap 144 and the surrounding host articular cartilage.

In a surgical procedure to implant an osteochondral plug according to an embodiment of the present invention, such as one of the plugs 120, 130 and 140 disclosed above, an articular cartilage lesion or defect is removed by cutting a bore in the defect area. An osteochondral plug according to an embodiment of the present invention, such as one of the plugs 120, 130 or 140 disclosed above, is then inserted into the bore to form (i) an interference fit between the subchondral bone portion of the plug and the sidewall of the bore, and (ii) a ring-shaped gap or trough between the upper cap portion of the plug and an adjacent portion of the sidewall of the bore. The trough is then filled with a desired amount of the aforementioned cartilage particle mixture. This arrangement retains the cartilage particle mixture in the bore for a predetermined period of time, to promote chondrocyte (and/or other cellular) migration into (i.e., from the adjacent host cartilage) and proliferation in the trough and to enhance tissue integration between the plug and the host surrounding articular cartilage. The cartilage particle mixture may also be placed directly on one or more surfaces of the plug during the surgical procedure.

The diameter, or lateral dimension, of the plugs illustrated in FIGS. 10-13 may be in a range from 1 mm to 30 mm, but is preferably 3 mm to 10 mm, which is small enough to fit through an endoscopic cannula, but large enough to minimize the number of plugs needed to fill large defects. This size provides good results at the recipient site and provides a more confluent articular cartilage surface. A surgeon may reduce the height of the plug by cutting and removing a bottom portion of the subchondral bone portion to match the depth of the bore, so that the cartilage cap of the plug will be even with the surface of the host articular cartilage.

The osteochondral plugs disclosed herein provide a less dense matrix, which allows more cells to enter the plug, once implanted in the bore. During surgery, the plug can be inserted arthroscopically or through an open incision.

The allograft cartilage particle mixture described below may be used in a prepackaged amount with the osteochondral plugs disclosed herein. For example, the cartilage particle mixture may be used to fill the trough of the embodiments illustrated in FIGS. 10-13 and to promote chondrocyte (and/or other cellular) migration into (i.e., from the adjacent host cartilage) and proliferation in the trough and to enhance tissue integration between the plug and the host surrounding articular cartilage. The base of the bore may be provided with the cartilage particle mixture as well, for example, when used with the embodiments illustrated in FIGS. 2-9, to promote chondrocyte (and/or other cellular) migration into and proliferation in the trough and to enhance tissue integration between the plug and the host surrounding articular cartilage.

It is also envisioned that the cartilage particles disclosed below and/or the osteochondral plug can be coated with a solution containing adeno-associated virus vectors (AAV), recombinant adeno-associated virus vectors (rAAV), or any other viral or non-viral vector. An AAV contains only two genes: (1) a rep gene which codes for proteins involved in DNA replication and (2) a cap gene which, by differential splicing, codes for the three proteins that make up the protein coat of the virus.

### Cartilage Particles

In one embodiment, the cartilage particles described herein are administrable as a stand-alone therapeutic treatment.

In one embodiment, the cartilage particles described herein are milled allograft cartilage particles. In one embodiment, allograft cartilage particles are milled, e.g., by use of a freeze-milling (i.e., freezer-milling) process wherein the cartilage is cryogenically frozen, for example by use of a liquefied gas freezing agent (e.g., liquid nitrogen or liquid helium) and ground into particles.

In another embodiment, an allograft osteochondral plug according to the present invention and as described above is surrounded with or placed adjacent to a mixture, such as paste or gel, that includes freeze-milled allograft cartilage particles. In one embodiment, the term “gel” refers to a mixture of freeze-milled cartilage particles in a biocompatible carrier having a viscosity which is less than and is less rigid than a mixture of freeze-milled cartilage particles referred to by the terms “putty” or “paste” and contains less cartilage by weight than putty or paste. The cartilage paste or gel components are believed to provide the environmental and biochemical cues to elicit a cellular healing response. For example, paste or gel components such as proteoglycans, collagen type II and other extracellular matrix components and their substituents may be present in greater concentration and/or bioavailability as a function of the processing of freeze-milled cartilage; e.g. freeze-milling cartilage may result in cartilage particles that are characterized as having greater exposure/bioavailability of different cytokines, growth factors, etc. to the surrounding environment. These available factors may then exert effects on cells that have infiltrated the plug from the surrounding host tissue and bleeding bone, synovium, etc. In one embodiment, the cells are chondrocytes. In one embodiment, the cells are capable of differentiation into chondrocyte lineage. In one embodiment, the cells are mesenchymal stem cells. Further examples include, without limitation, pluripotent stem cells; progenitor cells; mesenchymal stem and progenitor cells; stromal cells; and cartilage stem cells.

In one embodiment, the cartilage particles have a size ranging from about 10 microns (μm) to about 212 microns. In one embodiment, the cartilage particles have been reconstituted in saline solution (e.g., PBS), as discussed herein. Alternatively, the cartilage particles may be smaller than 10 microns (see below), and/or have a mean and/or median size distributed anywhere between 10 and 200 microns. The small size of the cartilage particles can increase the exposure or release of various growth factors due to the increased aggregate surface area of the particulate cartilage used, and can increase the capacity of the surrounding and infiltrating cells to attach to cartilage particles.

The cartilage particles may be irregularly shaped, and are passed through a sieve having 212 micron openings. While one dimension of each of the particles should be 212 microns or smaller in at least one dimension in order to fit through the sieve, certain other axis lengths of the same particles may have axis lengths longer than 212 microns, rendering the particles unable to pass through the sieve openings in that particular orientation. Several differently-sized cartilage particles are described in U.S. Pat. No. 7,067,123, and in U.S. patent application Ser. Nos. 12/043,001; 11/657,042; and 60/996,800, all of which are fully incorporated by reference herein in their entirety. The aforesaid dimension of such particles may be less than or equal to 212 microns; from about 5 microns to about 212 microns; from about 6 microns to about 10 microns; less than or equal to 5 microns; and/or less than or equal to 100 microns. In one embodiment, the aforesaid dimension of most of the particles is less than 100 microns.

The cartilage gel or paste provides the environment and necessary biochemical cues to elicit a healing response from the cells that have infiltrated the plug from the surrounding host tissue, synovium and/or bleeding bone that undergoes blood clotting and other reparative processes. In one embodiment, these biochemical cues include the exposure or release of various growth factors, as discussed herein.

The cartilage particles are preferably derived from allograft cartilage, such as allograft articular cartilage. For example, such cartilage particles may be composed at least partially of collagen type II and proteoglycans, which may provide a non-cellular matrix to support cell attachment and to facilitate extracellular matrix production. The cartilage particles may also be derived from fibrous cartilage, or a combination of hyaline and fibrous cartilage. Alternatively, autograft or xenograft cartilage may be used.

In an alternative embodiment, a gel or paste containing fibrous cartilage particles (e.g., derived from meniscus, tendons, ligaments, intervertebral disc, etc.) may be used for repairing defects in fibrous cartilage tissues (e.g., meniscus, tendons, ligaments, intervertebral disc, etc.) For example, defects in a meniscus may be repaired using a paste mixture containing cartilage particles derived from meniscus tissues.

In another embodiment, any of a number of tissues (e.g., meniscus, tendons, ligaments, skin, fascia, periosteum, muscle, fat, intervertebral disc, etc.) may be freeze-milled and subsequently utilized in defect repair and/or genesis of similar or physiologically unrelated tissues.

The starting material from which cartilage particles are derived may be lyophilized. In one embodiment, the starting material from which cartilage particles are derived will have been lyophilized prior to freeze-milling, so that their water content ranges from 0.1% to 8.0%. In another embodiment, the cartilage particles resulting from the freeze-milling process may be lyophilized again (i.e., re-lyophilized). In another embodiment, the cartilage particles resulting from the freeze-milling process may be rehydrated before re-lyophilization. The cartilage particles may range from about 15% to about 50%, by weight, of a gel or paste (in one embodiment, about 22%), and may be mixed with a carrier, which constitutes the remaining weight of the gel or paste. The carrier may have a composition of one or more of the following: phosphate buffered saline (PBS) solution, saline solution, sodium hyaluronate solution (HA) (molecular weight ranging from 7.0×10⁵ to 1.2×10⁶) and other suitable bioabsorbable carriers such as hyaluronic acid and its derivatives, gelatin, collagen, chitosan, alginate, Dextran, sterile water, carboxymethylcellulose (CMC), hydroxypropyl methylcellulose, or other polymers, blood or plasma. The cartilage particles can be processed to various particle sizes and the carrier can have different viscosities, depending on the desired consistency of the gel or paste. This cartilage gel or paste can be placed adjacent to the plug, as described above. The cartilage gel or paste enhances the tissue integration between the plug and adjacent host tissue. For example, the use of cartilage gel or paste in repairing an articular cartilage defect may result in production of new, well-organized articular cartilage tissue, accompanied by a restored “tidemark”.

### Endogenous And Exogenous Growth Factors

As discussed above, cartilage paste or gel components are believed to provide the environmental and biochemical cues necessary to elicit a cellular healing response. For example, cartilage that has been freeze-milled may have greater exposure/bioavailability of different endogenous cytokines, growth factors, etc., relative to the surrounding environment. These may include, without limitation, at least native FGF-2, IGF-1, TGF-β (including TGF-β1), BMP-2, and BMP-14 (GDF-5).

The cartilage particles may be provided alone, or may be combined with a plug during a surgical procedure according to the present invention, and may be provided to a medical practitioner without added cells or added growth factors. Such cartilage particles (alone and in combination with a plug) are themselves capable of supporting articular cartilage regeneration without addition of further materials.

As noted herein, one or more exogenous chondrogenic growth factor additives may be loaded into the bore adjacent to and/or absorbed into the plug of the present invention. Such growth factors may include recombinant or native or variant growth factors of FGF-2, FGF-5, FGF-7, FGF-9, FGF-11, FGF-21, TGF-β (including TGF-β1), BMP-2, BMP-4, BMP-7, BMP-14 (GDF-5), PDGF, VEGF, IGF-1, and bioreactive peptides such as Nell 1 (e.g., UCB1) and TP508. Additional growth factors which can be added include hepatocyte growth factor and platelet-derived growth factor. Other possible additives include human allogeneic or autologous chondrocytes, human allogeneic cells, human allogeneic or autologous bone marrow cells, human allogeneic or autologous stem cells, synovial cells, mesenchymal stem cells, pluripotent stem cells, mesenchymal stem and progenitor cells, stromal cells, cartilage stem cells, demineralized bone matrix, insulin, interleukin-1 receptor antagonist, Indian hedgehog, parathyroid hormone-related peptide, viral vectors for growth factor or DNA delivery, nanoparticles, platelet-rich plasma, fibrin clot, bioabsorbable polymers, hyaluronic acid, bone marrow aspirate, xenogenic chondrocytes and mesenchymal stem cells, naked DNA, and RNA. Any one or more of the above-listed additives may be absorbed or combined with the plug and/or the aforementioned cartilage particle gel or paste, or added directly to the bore and/or cartilage particles described herein. As an illustration, a chondrogenic growth factor may be adsorbed into the plug, or into the cartilage particle gel or paste, added to the bore adjacent the plug, or into both the plug and the cartilage particle gel or paste.

In one embodiment, the growth factor TGF-β is included as an activatable endogenous component and/or as an exogenous component (latent or active) in any of the embodiments disclosed herein.

In another embodiment, any member of the growth factor family FGF and/or a natural or recombinant variant thereof is included (as an endogenous component and/or as an exogenous component) in any of the embodiments disclosed herein. One description of a member of the FGF family's structure and physiological role (particularly relating to enhancing chondrogenesis and chondrogenesis lineage commitment from mesenchymal stem cells) is found in the article “FGF-2 Enhances the Mitotic and Chondrogenic Potentials of Human Adult Bone Marrow-Derived Mesenchymal Stem Cells” (L. A. Solchaga et al., Journal of Cellular Physiology 203:398-409 (2005)), which is incorporated herein by reference in its entirety. In one embodiment, FGF-2 binding enhances chondrocyte proliferation. In one embodiment, FGF-2 binding enhances chondrocyte differentiation. In one embodiment, FGF-2 binding increases chondrocyte aggregation. In one embodiment, FGF-2 binding increases development of chondrocyte-mediated creation of extracellular matrix. In one embodiment, FGF-2 binding increases proteoglycan synthesis. In one embodiment, FGF-2 binding mediates increased collagen type II/type I ratio as compared to control cells. In one embodiment, FGF-2 binding downregulates MAP kinase activities. In one embodiment, FGF-2 binding inhibits MAP kinase activities.

In another embodiment, freeze-milled cartilage particles having a size less than 212 microns (μm), preferably ranging from approximately 10 to 212 microns, are combined with a phosphate buffered saline (PBS) carrier and an exogenous fibroblast growth factor such as FGF-2 or a variant thereof in a therapeutically effective and/or efficacious dosage. This combination may be added to the bore so as to be adjacent the plug, as disclosed herein.

In another embodiment, any member of the growth factor family BMP is included (as an endogenous component and/or as an exogenous component) in any of the embodiments disclosed herein. One description of a member of the BMP family's structure and physiological role (particularly relating to initiating chondrogenesis and chondrogenesis lineage commitment from mesenchymal stem cells) is found in the article “BMP2 initiates chondrogenic lineage development of adult human mesenchymal stem cells in high-density culture” (B. Schmitt et al., Differentiation (2003) 71:567-577), incorporated herein by reference in its entirety. In one embodiment, BMP2 may be co-administered with TGF-β 3 so as to drive chondrocyte differentiation from MSCs (mesenchymal stem cells). In one embodiment, BMP2 may drive selective differentiation. In one embodiment, administration of BMP2 results in substantially no adipocyte or osteoclast cell differentiation. In one embodiment, BMP2 facilitates upregulation of COMP-type II collagen and cartilage oligomeric matrix protein synthesis. In another embodiment, BMP2 facilitates development of high density chondrocyte microenvironments, which may be important for cell-to-cell signaling so as to maintain chondrocyte lineage.

### Activation of Latent Endogenous Growth Factors

In one embodiment, the small size of the particulate cartilage can lead to increased activation of various latent forms of growth factors due to the increased aggregate and/or accessible surface area of the particulate cartilage used. Examples specific to TGF-β are described herein, but mechanical, physical and/or chemical activation processes described herein are applicable to a wide range of latent endogenous growth factors.

TGF-β is synthesized and secreted as a biologically inactive or “latent” complex. Activation must occur to release the mature, biologically active form of TGF-β, for signal transduction. The mechanism of activation of latent TGF-p in vivo is still not completely understood. It may occur by local acidification at the site of action or by endogenous and/or exogenous enzymatic activity, and may also involve integrins, thrombospondin, metalloproteases, plasmin, furin and other proteases. Latent TGF-β (L-TGF-β) can be activated in vitro by acid or alkaline solutions (pH 2 or pH 8, respectively), exposure to heat (e.g., 100° C.), or by treatment with chaotropic agents and substances like SDS and urea. In one embodiment, the molecular weight of TGF-β is reduced from 100 kD to 25 kD prior to or simultaneously with activation. Various physiological substances have been reported to activate L-TGF-β in in vitro studies. Some examples are serine protease, plasmin, other proteases such as endoglycosidase F, sialidase, neuraminidase, cathepsins B and D, calpain, and the glycoprotein, thrombospondin-1, all of which can convert L-TGF-β to biologically active TGF-β. In one embodiment, TGF-β1 is cleaved from the C-terminus of a disulfide-linked dimer of pro-TGF-β1 by a subtilsin-like pro-protein convertase protease. It is normally secreted as an inactive, or latent, complex. Although it is not always stated, the isoform most often described to be susceptible to the actions of the aforementioned substances is TGF-β1.

Increased exposure, release, or activation of various growth factors may also be attributable to pH-mediated physical and/or chemical changes to the tissue. In another embodiment, such pH-mediated physical and/or chemical changes resulting in exposure, release, or activation of various growth factors are attributable to an acidic pH (for example, pH 2). In another embodiment, such pH-mediated physical and/or chemical changes resulting in exposure, release, or activation of various growth factors is attributable to an alkaline pH (for example, pH 8).

Increased exposure, release, or activation of various growth factors may also be attributable to temperature-mediated physical and/or chemical changes to the tissue. In one embodiment, growth factor activation occurs at mammalian body temperature (e.g., 37° C.). In another embodiment, growth factor activation is inhibited at low temperatures (e.g., −40° C.) with a subsequent measurable increase in growth factor structural stability. In one embodiment, the physiological mechanism of release from latency is an important control for the regulation and localization of TGF-βactivity. In one embodiment, proteolysis of latent TGF-β is likely a part of the mechanism of release from latency.

Increased exposure, release, or activation of various growth factors may also be attributable to release of endogenous proteases and subsequent protease-mediated physical and/or chemical changes to the tissue. In one embodiment, the endogenous protease is serine protease. In one embodiment the endogenous protease is a cathepsin. In one embodiment, the endogenous protease is a sialidase. In another embodiment, the sialidase is a neuramidase. In another embodiment, the endogenous protease is an endoglycosidase. In another embodiment, the endoglycosidase is endoglycosidase F, retinoic acid, and/or transglutaminase.

Increased exposure, release, or activation of various growth factors may also be attributable to release of chaotropic agents and subsequent physical and/or chemical changes to the tissue.

In another embodiment, increased exposure, release, and/or activation of various growth factors is attributable to mechanical disruption of the freeze-milled cartilage, resulting in increased exposure of cartilage proteoglycans and other cartilage components to the outside environment.

Increased exposure, release, or activation of various endogenous growth factors may also be attributable to lyophilization of the freeze-milled cartilage, either before or after freeze-milling.

Increased exposure, release, or activation of various endogenous growth factors may also be attributable to conversion of one or more other growth factors from the latent stage.

Growth factor effects may be context-dependent; e.g., a growth factor that would drive osteogenesis in a vascularized microenvironment will drive chondrogenesis in an avascular environment.

In one embodiment, the growth factor isoform often found to be susceptible to the actions of the aforementioned substances and/or manipulations is latent TGF-β1. In another embodiment, the growth factor isoforms often found to be susceptible to the actions of the aforementioned substances and/or manipulations are L-TGF-β2 and 3.

### Activation Of Exogenous Growth Factors

The cartilage particle gel or paste can also contain exogenous growth factors and/or growth factor activators. The levels of these growth factors may be similar to or greater than the levels of endogenous growth factors in intact cartilage. Exogenous growth factors and/or growth factor activators can also be combined with the cartilage particles. In one embodiment, the cartilage particles are mixed with a growth factor in an aqueous vehicle, after which the cartilage particles can either be lyophilized and stored dry at room temperature or frozen. Alternatively, the cartilage particles/growth factor mixture may be used immediately. In one embodiment, the mixture containing the cartilage particles and growth factor can be lyophilized for storage. In one embodiment, the lyophilized cartilage particles and growth factor may have a residual water content ranging from 0.1% to 8.0% by weight.

In another embodiment, the activatable exogenous growth factor can be any one of a variety of growth factors known to promote wound healing, cartilage and/or bone development (e.g., TGF-β).

In another embodiment, the activating agent used to solubilize the growth factor and/or adsorb it into the cartilage particles (or alternately to activate endogenous growth factors present in the freeze-milled cartilage particles) can be saline, water, PBS, Ringers, any agent capable of pH modification or proteolytic activity, etc.

In another embodiment, the resulting enhanced cartilage particles can contain levels of growth factors that are greater than that found in intact cartilage. In another embodiment, the cartilage particle mixture can placed adjacent the cartilage cap of the plug (e.g., on an upper surface of the cartilage cap of one of the embodiments of the plug disclosed herein, and/or placed in the bore formed in a patient's articular cartilage defect area, and/or in the trough formed between the bore sidewall and the cartilage cap of one of the embodiments of the plug disclosed herein).

In another embodiment, cells which have been collected from the patient or grown outside the patient can be added to the plug during or after implantation of the plug into the bore. Such cells include, for example, allogenic or autologous bone marrow cells, stem cells and chondrocytes. A therapeutically effective cellular density may be utilized. In one embodiment, the cellular density of the cells preferably ranges from 1.0×10⁸ to 5.0×10⁸ cells/cc of paste or gel mixture. In one embodiment, the cellular density of the cells preferably ranges from 5.0×10⁶ to 1.0×10⁸ cells/cc of paste or gel mixture.

In another embodiment, any of the methods of the instant invention can be utilized to repair or stimulate growth of meniscus, muscle, tendons, ligaments, skin, periosteum and fat tissue. In another embodiment, meniscus, muscle, tendons, ligaments, skin, periosteum and/or fat tissue may itself be particularized and subsequently utilized to repair analogous and/or nonanalogous tissues.

The following examples further illustrate aspects of the various embodiments of the present invention.

### Example 1

### Complete Interference-Fit Osteochondral Plug

A non-viable or decellularized osteochondral plug including a subchondral bone base and an overlying cartilage cap, according to one of the embodiments disclosed herein and illustrated in FIGS. 2-9, was treated with a solution or variety of solutions such as hyaluronidase (type IV-S), trypsin and a chloroform/methanol solution to remove the cellular debris and the proteoglycans, as noted in the treatment described above. It is believed that this removal provides signaling to stimulate the surrounding chondrocytes to proliferate and form new proteoglycans and other factors producing new cartilage matrix. The plug is then subjected to an antibiotic soak and milled to a desired configuration (e.g., as illustrated in FIG. 3, 5, 7 or 9) to have an interference fit with the blind bore to be cut in the patient's tissue.

### Example 2

### Partial Interference-Fit Osteochondral Plug

A non-viable or decellularized osteochondral plug including a subchondral cylindrical bone base and an overlying smaller diameter cylindrical cartilage cap cut from the original plug, according to one of the embodiments disclosed herein and illustrated in FIGS. 10-13, was treated with a solution or variety of solutions such as hyaluronidase (type IV-S), trypsin and a chloroform/methanol solution to remove the cellular debris as well as the proteoglycans, as noted in the treatment described above. It is believed that this removal provides signaling to stimulate the surrounding chondrocytes to proliferate and form new proteoglycans and other factors producing new matrix. The plug is then subjected to an antibiotic soak and milled to a desired configuration (e.g., as illustrated in FIG. 11, 12 or 13) so that the subchondral bone portion will have an interference fit with the blind bore to be cut in the patient's tissue.

### Example 3

### Tissue Extraction and Particularization

A process of cartilage particle extraction may be applied to any of a number of different soft tissue types (for example, meniscus tissue). In one embodiment, cartilage is recovered from deceased human donors, and the tissue is treated with a soft tissue process.

Fresh articular cartilage is removed from a donor using a scalpel, taking care to remove the cartilage so that the full thickness of the cartilage is intact (excluding any bone). Removed cartilage is then packaged in double kapak bags for storage until ready to conduct chemical cleaning of the allograft tissue. In one example, the cartilage can be stored in the refrigerator for 24-72 hours or in the freezer (e.g., at a temperature of −70° C.) for longer-term storage.

Chemical cleaning of cartilage tissue is then conducted according to methods known by those skilled in the art. Subsequent to chemical cleaning, the cartilage is lyophilized, so as to reduce the water content of the cartilage tissue to within the range of about 0.1% to about 8.0%. The lyophilized cartilage is then dried.

Subsequent to the initial lyophilization, the cartilage is freeze-milled, wherein the cartilage is frozen (for example, with liquid nitrogen as a freezing agent) and ground into particles. The cartilage particles are sieved, for example, through a 212 micron sieve.

Next, the lyophilized, freeze-milled cartilage particles are processed into a gel or paste through a combination of the freeze-milled cartilage particles with PBS. Exogenous growth factors are optionally added at this stage, and the cartilage particle/exogenous growth factor/PBS mixture is optionally left to equilibrate. Optionally, growth factor may be added to the cartilage particles without or prior to subsequent processing into a gel or paste. The cartilage paste may be optionally lyophilized again subsequent to the addition of growth factors.

### Example 4

### Extraction of Proteins from Human Cartilage Using Extraction and Subsequent Dialysis

In another example, growth factors may be physically and/or chemically isolated from cartilage particles, and dialyzed using a suitable agent. The growth factors are thereby isolated for subsequent analysis and/or quantification. In one embodiment, 0.3 g of cartilage particles were weighed out for each donor. The cartilage particles were transferred to tubes containing 5 ml of extraction solution (4M Guanidine HCl in Tris HCl). The cartilage particles were incubated at 4° C. on the orbital shaker at 60 RPM for 24 hours, followed by dialysis (8k MWCO membrane dialysis tube) in 0.05M TrisHCl or PBS for 15 hrs. at 4° C. The dialysis solution was then replaced and the dialysis continued for another 8 hrs. at 4° C. The post-dialysis extracts were stored at −70° C. until the ELISA was run.

### Example 5

### ELISA Analysis of Endogenous Growth Factors in Cartilage Particles

The quantities and concentrations of various endogenous growth factors isolated from cartilage may be assessed utilizing ELISA technology. ELISA analysis may be conducted using any available ELISA protocol, including but not limited to R&D System's ELISA kit and ProMega's TGF-β Emax™ ImmunoAssay System.

### Example 6

### Quantification of Endogenous Growth Factors Present in Processed Cartilage

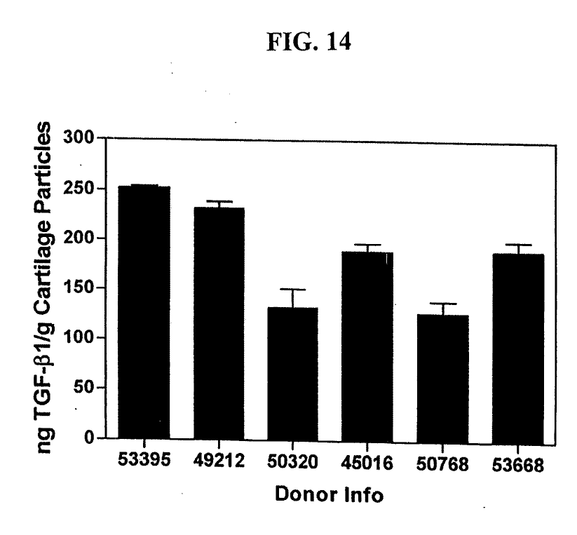

0.3 g of freeze-milled, processed cartilage particles were weighed out for each donor. The cartilage particles were transferred to tubes containing 5 ml of extraction solution (4M Guanidine HCl in TrisHCl). The cartilage particles were incubated at 4° C. on the orbital shaker at 60 rpm for 24 hrs, followed by dialysis (8k MWCO membrane dialysis tube) in 0.05M TrisHCl or PBS for 15 hrs at 4° C. The dialysis solution was then replaced and the dialysis continued for another 8 hrs at 4° C. The post-dialysis extracts were stored at −70° C. until the ELISA was run. Notably, the above protocol can also be utilized in order to determine the total endogenous growth factor concentration present in a device of the instant invention. FIG. 14 demonstrates the relative concentration of endogenous total TGF-β1 found in freeze-milled cartilage particles of the present invention derived from various subjects.

### Example 7

### Increased Availability of Endogenous TGF-β1 from Freeze-Milled Cartilage

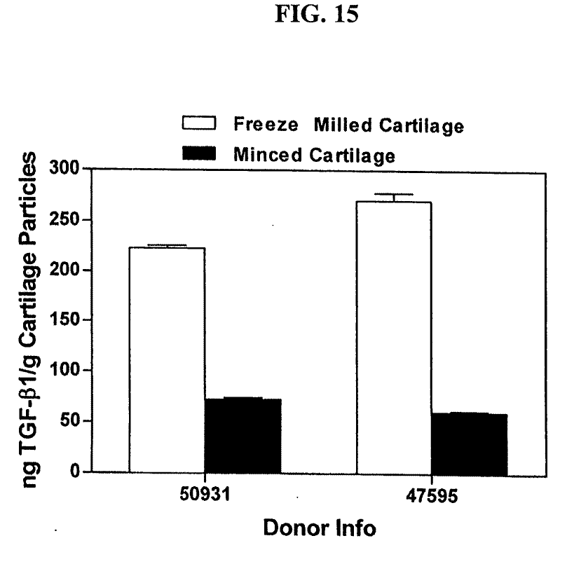

In order to assess the relative amounts of endogenous TGF-β1 accessible via guanidine extraction, the guanidine extraction of endogenous TGF-β1 from minced (e.g., not freeze-milled) cartilage pieces was compared to the guanidine extraction of TGF-β1 from freeze-milled, processed cartilage particles. Increased amounts of endogenous TGF-β1 may be extractable from freeze-milled cartilage particles, as opposed to minced (e.g., not freeze-milled) cartilage pieces. This may be attributable to the increased surface area of the freeze-milled cartilage particles. For example, the fracture planes; three-dimensional shape of the particles; and resultant increased surface area-dependent may enhance the release of the cartilage growth factor(s) or other substances from the particles, or the accessibility of growth factors to surrounding cells. This may influence bioavailability of endogenous growth factors and activation of latent endogenous growth factors. Furthermore, the avoidance of elevated temperatures during processing may facilitate the production of particles having high chondrogenic activity by facilitating substantial preservation of extracellular matrix components. For example, preservation of the required tertiary or quaternary folding structures of endogenous growth factors or other proteins in tissue subjected to freeze-milling may occur. FIG. 15 provides relative amounts of growth factor isolated from minced cartilage and from freeze-milled cartilage of the present invention.

### Example 8

### Quantification of Total Endogenous FGF-2 Present in Processed Cartilage Particles

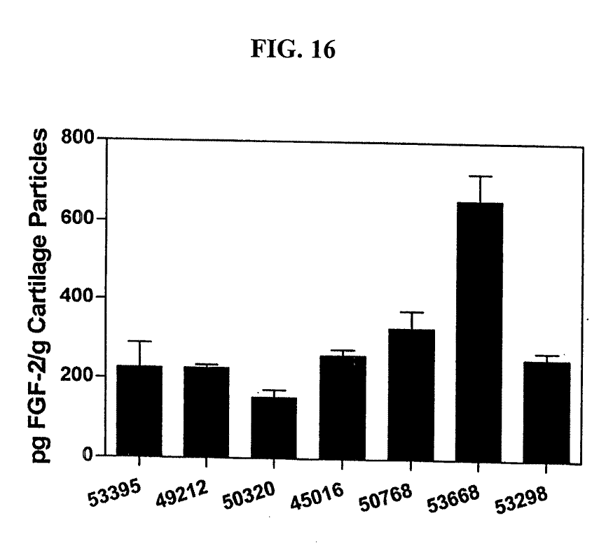

FIG. 16 demonstrates the relative concentration of endogenous FGF-2 found in freeze-milled cartilage particles of the present invention that were prepared in accordance with the process illustrated in Example 3 of the present application, after being harvested from various tissue donors.

### Example 9

### Quantification of Total Endogenous BMP-2 Present in Processed Cartilage Particles

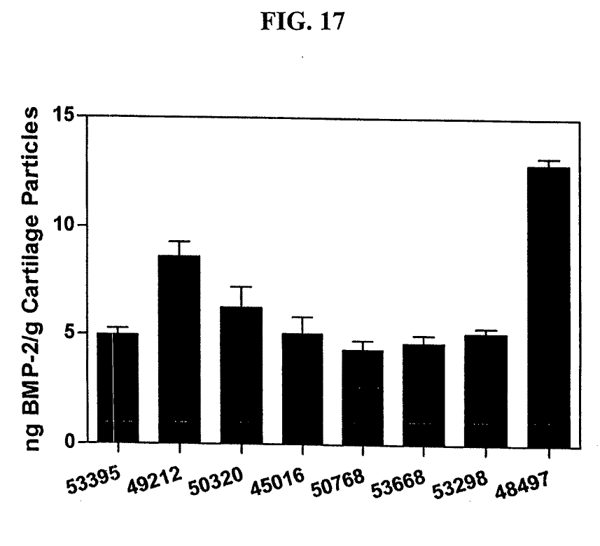

FIG. 17 demonstrates the relative concentration of endogenous BMP-2 found in freeze-milled cartilage particles of the present invention that were prepared in accordance with the process illustrated in Example 3 of the present application, after being harvested from various tissue donors.

### Example 10

### Quantification of Total Endogenous GDF-5/BMP-14 Present in Processed Cartilage Particles

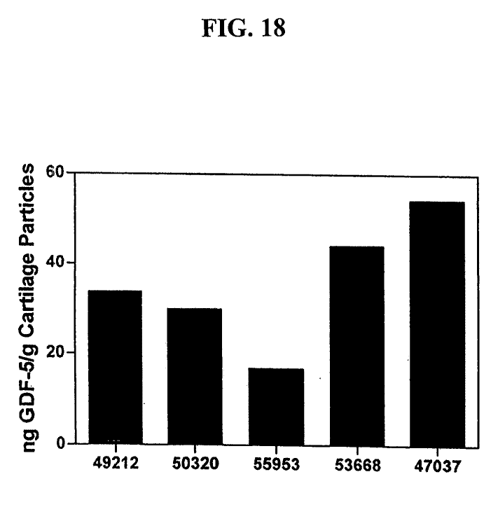

FIG. 18 demonstrates the relative concentration of endogenous GDF-5/BMP-14 found in freeze-milled cartilage particles of the present invention that were prepared in accordance with the process illustrated in Example 3 of the present application, after being harvested from various tissue donors.

### Example 11

### Quantification of Total Endogenous IGF-1 Present in Processed Cartilage Particles

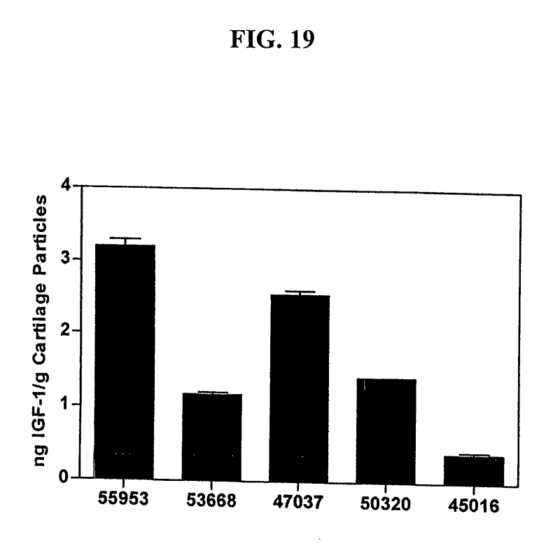

FIG. 19 demonstrates the relative concentration of endogenous IGF-1 found in freeze-milled cartilage particles of the present invention that were prepared in accordance with the process illustrated in Example 3 of the present application, after being harvested from various tissue donors.

As shown in the included tables and figures, processed cartilage particles, minced cartilage, and native cartilage all retain a concentration of endogenous TGF-β1. Such concentration of TGF-β1 is more bioavailable in the freeze-milled particles described herein. Freeze-milled cartilage particles as described herein also retain a concentration of endogenous BMP-2; BMP-14/GDF-5; IGF-1; and FGF-2.

### Example 12

### Relative Efficacy of Various Cartilage Paste and Clinical Standard Methods in Particular Cartilage Reconstruction In Vivo (Microfracture)

In another embodiment, a study was conducted to determine the cartilage healing potential of the four cartilage paste preparations listed in TABLE 1 below. Chondral defects were created on the medial femoral condyle (6 mm diameter) and trochlear sulcus (5 mm diameter) followed by microfracture and implantation of the cartilage paste.

TABLE 1

| | | | # of | Study |
|---|---|---|---|---|
| Implant | Procedure | Splint Time | Goats | Time |
| Particles in PBS | Microfracture | 3, 7, or 14 | 3 | 6 weeks |
| | | days | | |
| Particles in Hy | Microfracture | 3, 7, or 14 | 3 | 6 weeks |
| | | days | | |
| Particles in Hy + | Microfracture | 3, 7, or 14 | 3 | 6 weeks |
| Insulin | | days | | |

All subjects exhibited some circumferential healing in one or more of the defects. This ranged from 0 to 75% in the medial femoral condyle lesion areas being filled with repair tissue, and from 0 to 90% in the trochlear sulcus lesion areas being filled with repair tissue.

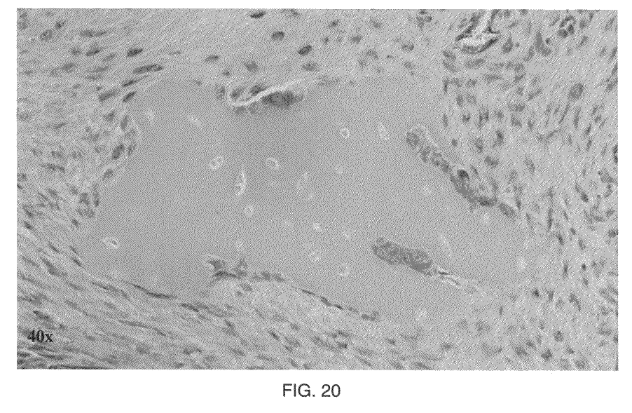

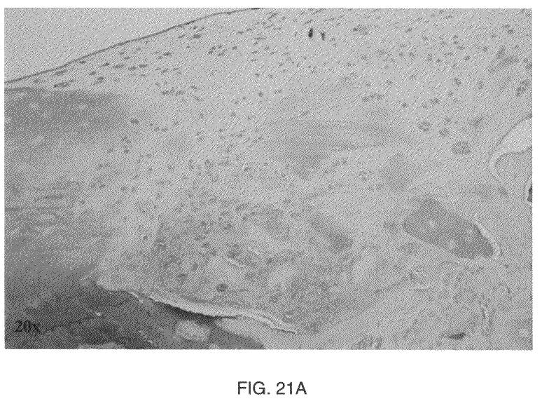

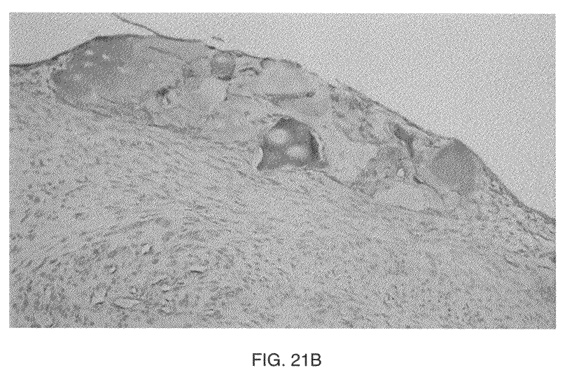

Histologically, it was observed that where the cartilage paste was retained in the defect, and that the repair tissue was positive for glycosaminoglycans (GAG) and collagen II, which indicated the presence of articular cartilage. This occurred most frequently around the edges of the defect where the paste had a better chance of staying in place. See FIG. 20 (demonstrating infiltration of lacunae by chondrocytes) and FIGS. 21A-B (residual and new collagen type II, respectively, visualized via immunohistochemistry).

Examples 13-16 below include data from a particle size analysis conducted on a sample of allograft cartilage particles from four different tissue donors in a dry state. An analysis of cartilage particles from the donors was also conduced after the particles were mixed with one of the following carriers: hyaluronic acid (Hy) paste, PBS and Ringers. The cartilage particles in the carriers were measured at 1 hour, 8 hours and 24 hours.

The particle size analysis was conducted on a Malvern MasterSizer Laser diffractor (Malvern Instruments Ltd., Worcestershire, United Kingdom) that calculated a volume distribution from the laser diffraction pattern. The raw scatter data was then processed using a complex algorithm resulting in an equivalent spherical diameter for the particles. The equivalent spherical diameter of an irregularly-shaped object, such as the particles, is the diameter of a sphere of equivalent volume as the object.

Of the dry cartilage particles that were analyzed, at least 95% of the particles had an equivalent spherical diameter of less than 100 microns, while at least 90% of the particles had an equivalent spherical diameter of less than 60 microns. The cartilage particles in the carriers had larger equivalent spherical diameters than those of the dry particles, which may have been attributable to the swelling of the particles in the carriers and/or to the agglomeration of the particles when mixed with the carriers. Based on the results of the analysis, the allograft cartilage particles were concluded to have equivalent spherical diameters of less than 100 microns.

### Example 13

### Particle Size Analysis of Cartilage Particles (Donor #1)

Cartilage particles derived from a 30 year old male tissue donor were pre-weighed and sent to Particle Technology Labs, Ltd. (Downers Grove, Ill.) for evaluation on the microscope using Image-Pro (Bethesda, Md.) analysis to determine their particle size dry. The cartilage was lyophilized and freeze-milled prior to evaluation. 0.2 grams of cartilage particles were set aside for dry analysis.

A total of 4,242 cartilage particles were analyzed. The equivalent spherical diameter of the particles ranged from less than 31.95 microns to 351.44 microns. According to the analysis, 2.59% of the particles had an equivalent spherical diameter greater than 95.85 microns, and 88.99% of the particles had an equivalent spherical diameter less than 31.95 microns. The arithmetic mean equivalent spherical diameter was 26.06 microns. The median equivalent spherical diameter was 11.30 microns. The mode equivalent spherical diameter was 31.95 microns. The data is presented in Table 2 below.

TABLE 2
Cartilage Particles Size Analysis Results for Donor #1

| Cumulative % | Equivalent Spherical | Equivalent Spherical |
|---|---|---|
| Indicated Size | Diameter (microns) | Volume (cubic microns) |
| 88.99% | 31.95 | 17,095.24 |
| 94.81% | 63.9 | 136,633.6 |
| 97.41% | 95.85 | 461,282.74 |
| 98.42% | 127.8 | 1,093,068.8 |
| 99.15% | 159.75 | 2,135,300.9 |
| 99.48% | 191.69 | 3,689,107.1 |
| 99.72% | 223.64 | 5,857,379.7 |
| 99.83% | 255.59 | 8,744,550.3 |
| 99.93% | 287.54 | 12,449,438 |
| 100.00% | 319.49 | 17,079,200 |
| 100.00% | 351.44 | 22,730,474 |

### Example 14

### Particle Size Analysis of Cartilage Particles (Donor #2)

Cartilage particles derived from a 50 year old female tissue donor were pre-weighed and sent to Particle Technology Labs, Ltd. (Downers Grove, Ill.) for evaluation on the microscope using Image-Pro (Bethesda, Md.) analysis to determine their particle size dry. The cartilage was lyophilized and freeze-milled prior to evaluation. 0.22 grams of cartilage particles were set aside for dry analysis.

A total of 2,174 cartilage particles were analyzed. The equivalent spherical diameter of the particles ranged from less than 24.62 microns to 270.79 microns. According to the analysis, 3.5% of the particles had an equivalent spherical diameter greater than 98.47 microns, and 88.22% of the particles had an equivalent spherical diameter less than 24.62 microns. The arithmetic mean equivalent spherical diameter was 24.04 microns. The median equivalent spherical diameter was 8.700 microns. The mode equivalent spherical diameter was 24.62 microns. The data is presented in Table 3 below.

TABLE 3
Cartilage Particles Size Analysis Results for Donor #2

| Cumulative % | Equivalent Spherical | Equivalent Spherical |
|---|---|---|
| Indicated Size | Diameter (microns) | Volume (cubic microns) |
| 88.22% | 24.62 | 7,814.82 |
| 92.73% | 49.23 | 62,518.57 |
| 95.08% | 73.85 | 211,000.19 |
| 96.50% | 98.47 | 500,148.59 |
| 97.56% | 123.08 | 976,376.67 |
| 98.48% | 147.70 | 1,687,316 |
| 99.17% | 172.32 | 2,679,550.8 |
| 99.49% | 196.93 | 3,999,970 |
| 99.68% | 221.55 | 5,695,462.5 |
| 100.00% | 246.17 | 7,812,917.4 |
| 100.00% | 270.79 | 10,399,223 |

### Example 15

### Particle Size Analysis of Cartilage Particles (Donor #3)

Cartilage particles derived from a 39 year old female tissue donor were pre-weighed and sent to Particle Technology Labs, Ltd. (Downers Grove, Ill.) for evaluation on the microscope using Image-Pro (Bethesda, Md.) analysis to determine their particle size dry. The cartilage was lyophilized and freezer milled prior to evaluation. 0.2 grams of cartilage particles were set aside for dry analysis.

A total of 2,667 cartilage particles were analyzed. The equivalent spherical diameter of the particles ranged from less than 30.01 microns to 330.13 microns. According to the analysis, 2.32% of the particles had an equivalent spherical diameter greater than 90.04 microns, and 91.45% of the particles had an equivalent spherical diameter less than 30.01 microns. The arithmetic mean equivalent spherical diameter was 23.36 microns. The median equivalent spherical diameter was 10.61 microns. The mode equivalent spherical diameter was 30.01 microns. The data is presented in Table 4 below.

TABLE 4
Cartilage Particles Size Analysis Results for Donor #3

| Cumulative % | Equivalent Spherical | Equivalent Spherical |
|---|---|---|
| Indicated Size | Diameter (microns) | Volume (cubic microns) |
| 91.45% | 30.01 | 14,139 |
| 95.84% | 60.02 | 113,225.15 |
| 97.68% | 90.04 | 382,262.23 |
| 98.65% | 210.08 | 4,855,221.6 |
| 99.21% | 150.06 | 1,769,496.7 |
| 99.59% | 180.07 | 3,058,097.8 |
| 99.65% | 210.08 | 4,855,221.6 |
| 99.74% | 240.09 | 7,248,220.7 |
| 99.89% | 270.11 | 10,321,080 |
| 100.00% | 300.12 | 14,155,974 |
| 100.00% | 330.13 | 18,842,971 |

### Example 16

### Particle Size Analysis of Cartilage Particles (Donor #4)

Cartilage particles derived from a 77 year old male tissue donor were pre-weighed and sent to Particle Technology Labs, Ltd. (Downers Grove, Ill.) for evaluation on the microscope using Image-Pro (Bethesda, Md.) analysis to determine their particle size dry. The cartilage was lyophilized and freezer milled prior to evaluation. 0.22 grams of cartilage particles were set aside for dry analysis.

A total of 3,678 cartilage particles were analyzed. The equivalent spherical diameter of the particles ranged from less than 28.31 microns to 311.45 microns. According to the analysis, 0.6% of the particles had an equivalent spherical diameter greater than 84.94 microns, and 96.87% of the particles had an equivalent spherical diameter less than 28.31 microns. The arithmetic mean equivalent spherical diameter was 15.45 microns. The median equivalent spherical diameter was 10.01 microns. The mode equivalent spherical diameter was 28.31 microns. The data is presented in Table 5 below.

TABLE 5
Cartilage Particles Size Analysis Results for Donor #4

| Cumulative % | Equivalent Spherical | Equivalent Spherical |
|---|---|---|
| Indicated Size | Diameter (microns) | Volume (cubic microns) |
| 96.87% | 28.31 | 11,894.19 |
| 98.69% | 56.63 | 95,153.48 |
| 99.40% | 84.94 | 320,916.24 |
| 99.76% | 113.25 | 760,824.7 |
| 99.84% | 141.57 | 1,486,143.2 |
| 99.92% | 169.88 | 2,567,330 |
| 99.95% | 198.19 | 4,077,236.3 |
| 99.97% | 226.51 | 6,086,597.6 |
| 99.97% | 254.82 | 8,664,738.6 |
| 100.00% | 283.13 | 11,886,626 |
| 100.00% | 311.45 | 15,822,014 |

While a number of embodiments of the present invention have been described, it is understood that these embodiments are illustrative only, and not restrictive, and that many modifications and/or alternative embodiments may become apparent to those of ordinary skill in the art. For example, any steps may be performed in any desired order (and any desired steps may be added and/or any desired steps may be deleted). Therefore, it will be understood that the appended claims are intended to cover all such modifications and embodiments that come within the spirit and scope of the present invention.

We claim:

1. In combination, a mixture including lyophilized, freeze-milled cartilage particles, at least a majority of which have a dimension, when dry, that does not exceed 212 microns; and a sterile allograft osteochondral plug, said plug including a subchondral bone portion and an integral overlying cartilage cap which has been treated to remove cellular debris, chondrocytes and proteoglycans, at least a portion of said plug having a lateral dimension selected to form an interference fit against a tissue layer exposed as a result of a bore formed in a defect area in articular cartilage of a host, wherein said cartilage particle mixture is placed adjacent at least a portion of said plug for promoting cartilage cell migration into and proliferation in the bore and for enhancing tissue integration between said plug and host tissue, when said plug is inserted into the bore.

2. The combination as claimed in claim 1, wherein said dimension is in a range of from about 5 microns to about 212 microns.

3. The combination as claimed in claim 2, wherein said dimension is in a range of from about 6 microns to about 10 microns.

4. The combination as claimed in claim 1, wherein said dimension is less than or equal to about 5 microns.

5. The combination as claimed in claim 1, wherein said dimension is less than or equal to 100 microns.

6. The combination as claimed in claim 1, wherein said cartilage particles are derived from articular cartilage.

7. The combination as claimed in claim 1, wherein said cartilage particle mixture further comprises a chondrogenic stimulating factor.

8. The combination as claimed in claim 7, wherein said chondrogenic stimulating factor is one or more of a group consisting of growth factors, bioreactive peptides, recombinant, native growth factors, human allogenic or autologous chondrocytes, human allogenic or autologous bone marrow cells, stem cells, demineralized bone matrix, insulin, insulin-like growth factor-1, transforming growth factor-B, interleukin-1 receptor antagonist, hepatocyte growth factor, platelet-derived growth factor, Indian hedgehog and parathyroid hormone-related peptide or bioactive glue.

9. The combination as claimed in claim 8, wherein said growth factors are selected from a group consisting of FGF-2, FGF-5, FGF-7, FGF-9, FGF-11, FGF-21, IGF-1, TGF-β, BMP-2, BMP-4, BMP-7, BMP-14, GDF-5, PDGF and VEGF.

10. The combination as claimed in claim 1, wherein said cartilage particle mixture further comprises a biocompatible carrier.

11. The combination as claimed in claim 9, wherein said biocompatible carrier comprises one or more of a group consisting of sodium hyaluronate, hyaluronic acid and its derivatives, gelatin, collagen, chitosan, alginate, buffered PBS, Dextran, polymers, Ringers, and synthetic and peptide based hydrogels.

12. The combination as claimed in claim 1, wherein said allograft osteochondral plug has been lyophilized so that its water content ranges from about 0.1% to about 8.0%.

13. The combination as claimed in claim 1, wherein said cartilage cap includes a first cap portion, which is located proximal to said subchondral bone portion of said plug, said first cap portion having a diameter the same as that of said subchondral bone portion of said plug, and a second cap portion, which is located remote from said subchondral bone portion of said plug, said second cap portion having a diameter less than that of said subchondral bone portion of said plug, said first and second cap portions being separated by an annular step which forms a ring-shaped gap positionable alongside a cartilage layer exposed as a result of the bore, said gap being sized and shaped so as to receive said cartilage particle mixture for promoting cartilage cell migration and proliferation in said gap and for enhancing tissue integration between said plug and host tissue, when said plug is inserted into the bore.

14. The combination as claimed in claim 13, wherein said first cap portion has a cylindrical shape, and said second cap portion has a cylindrical shape.

15. The combination as claimed in claim 13, wherein said first cap portion has a frustum conical shape and said second cap portion has a cylindrical shape.

16. The combination as claimed in claim 15, wherein said first cap portion includes a small diameter end positioned adjacent said second cap portion, and a large diameter end positioned adjacent said subchondral bone potion.

17. The combination as claimed in claim 13, wherein when said ring-shaped gap has a width in a range of from about 10 microns to about 1,000 microns.

18. The combination as claimed in claim 13, wherein when said ring-shaped gap has a width in a range of from about 100 microns to about 500 microns.

19. A method of repairing an articular cartilage defect of a patient, said method comprising the steps of:
providing a sterile allograft osteochondral plug, said plug including a subchondral bone portion and an integral overlying cartilage cap which has been treated to remove cellular debris, chondrocytes and proteoglycans;
forming a bore in an articular cartilage defect area of the patient, the bore exposing a subchondral bone layer and a cartilage layer in the defect area;
inserting a mixture into the bore, the mixture including lyophilized, freeze-milled cartilage particles, at least a majority of which have a dimension, when dry, that does not exceed 212 microns; and
inserting the plug into the bore so that at least a portion of the plug forms an interference fit against the exposed tissue of the patient, and at least a portion of the plug is adjacent the lyophilized, freeze-milled cartilage particle mixture, to promote cartilage cell migration into and proliferation in the bore and to enhance tissue integration between the plug and the patient's tissue.

20. A method of producing a mixture including freeze-milled cartilage particles for the repair of articular cartilage defects, the method comprising the steps of:
recovering allograft cartilage tissue from a human donor;
treating the allograft cartilage tissue for bioburden reduction;
chemically cleaning the allograft cartilage tissue;
lyophilizing the allograft cartilage tissue;
freeze-milling the allograft cartilage tissue into particles;
sieving the freeze-milled cartilage particles through a 212 micron sieve, such that each of the freeze-milled cartilage particles has a dimension, when dry, that does not exceed 212 microns; and
combining the freeze-milled cartilage particles with a biocompatible carrier.
